(19) United States
(12) Patent Application Publication
(10) Pub. No.: US 2026/0162808 A1
(43) Pub. Date: Jun. 11, 2026

(54) PROVIDING A COMPARISON DATA SET

(71) Applicant: Siemens Healthineers AG, Forchheim (DE)

(72) Inventors: Annette Birkhold, Stuttgart (DE); Markus Kowarschik, Erlangen (DE); Michael Manhart, Fürth (DE)

(21) Appl. No.: 19/411,261

(22) Filed: Dec. 6, 2025

(30) Foreign Application Priority Data
Dec. 6, 2024 (DE) 10 2024 211 680.7

(51) Int. Cl. G16H 30/40; G06V 10/75; G06V 10/764; G06V 40/10

(52) U.S. Cl. CPC G16H 30/40; G06V 10/751; G06V 10/764; G06V 40/15; G06V 2201/03

(57) ABSTRACT

A method for providing a comparison data set includes providing a time-resolved first data set including a plurality of first data points that have time intensity curves of a first contrast agent flow in a region of interest in a first time period, providing a time-resolved second data set including a plurality of data points that have time intensity curves of a second contrast agent flow in the region of interest in a second time period, and spatially and temporally registering the first data set and the second data set. The areas of the region of interest that are mapped in the data sets are classified into contrasted vessel segments and tissue region. A deviation between the tissue regions of the data sets is identified by comparison of the time intensity curves, and the comparison data set is provided based on the registered first and second data sets. The comparison data set contains at least one parameter characterizing the deviation.

This application claims the benefit of German Patent Application No. DE 10 2024 211 680.7, filed on Dec. 6, 2024, which is hereby incorporated by reference in its entirety.

### BACKGROUND

The present embodiments relate to a method for providing a comparison data set, a provisioning unit, a medical imaging device, and a computer program product.

It is common practice in interventional radiology to employ vessel embolization methods in order to change (e.g., reduce) a blood perfusion in certain tissue regions and/or to use methods that lead to a positive change in blood perfusion (e.g., a thrombectomy, a stenting of arterial stenoses, a treatment of vasospasms, and/or embolization of arteriovenous malformations). This may be the intention in a variety of treatments, such as, for example, in thrombectomy, tumor embolization, middle meningeal artery embolization, or migraine treatment. In a thrombectomy, an unintended change in tissue perfusion may occur due to distal embolisms.

Identifying and quantifying such changes in tissue perfusion with the aid of interventional imaging methods constitutes a major challenge. Conventional methods such as 3D perfusion imaging are often time-consuming and are associated with a relatively high X-ray dose. Further, they often provide no direct way of comparison between a state before and a state after an intervention. Other approaches are based, for example, on 2D digital subtraction angiography (DSA) image series and enable a color-coded representation of respective contrast intensities. However, these methods often deliver no direct measurement of the change in tissue perfusion in comparison with the initial state.

A further problem is that the existing methods are often not able to represent the perfusion changes already in the course of the intervention (e.g., in real time). This may make it more difficult for an interventional radiologist to respond immediately to unwanted changes or to judge the success of a treatment directly.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved identification and quantification of perfusion changes in a region of interest of an examination subject is provided.

Independent of the grammatical term usage, individuals with male, female, or other gender identities are included within the term.

In a first aspect, the present embodiments relate to a method for providing a comparison data set. In a first act, a time-resolved first data set is provided that includes a plurality of first data points, each having a time intensity curve. The time intensity curves of the first data points map a first contrast agent flow in a region of interest of an examination subject in a first time period. In a further act, a time-resolved second data set is provided that includes a plurality of second data points, each having a time intensity curve. The time intensity curves of the second data points map a second contrast agent flow in the region of interest of the examination subject in a second time period. In a further act, a spatial and temporal registration of the first data set and the second data set is performed. In a further act, areas of the region of interest that are mapped in the registered first data set and the registered second data set are classified into first areas that include contrasted vessel segments, and second areas that include a tissue region. In a further act, a deviation between the second areas of the registered first data set and the registered second data set is identified based on a comparison of the time intensity curves of the registered first data set and the registered second data set that map the second areas. In a further act, the comparison data set is provided based on the registered first data set and the registered second data set. The comparison data set includes at least one parameter characterizing the deviation.

In this process, the above-described acts of the method of the present embodiments for providing a comparison data set may be performed sequentially and/or at least to some extent concurrently. Further, the acts of the method of the present embodiments may be at least in part (e.g., completely) computer-implemented.

The providing of the first data set may include a receiving and/or a generation (e.g., a reconstruction) of the first data set. The providing of the second data set may also include a receiving and/or a generation (e.g., a reconstruction) of the second data set.

The receiving of the first data set and/or the second data set may, for example, include an acquisition and/or readout of a computer-readable data memory and/or a receiving from a data storage unit (e.g., a database). Further, the first data set and/or the second data set may be provided by a provisioning unit of one or more medical imaging devices (e.g., of the same or different imaging modalities). The at least one medical imaging device may be configured, for example, as a magnetic resonance tomography system (MRT) and/or computed tomography system (CT) and/or medical X-ray machine and/or positron emission tomography system (PET) and/or ultrasound device. For example, the first data set and the second data set may have been acquired using substantially the same acquisition parameters (e.g., of an acquisition geometry, such as angulation and/or imaging parameters) and/or injection parameters (e.g., of the respective contrast agent flow, such as of an injection rate). A comparability of the data sets may be improved by this.

Alternatively or in addition, the first data set may be generated (e.g., reconstructed) from preacquired first image data. Analogously thereto, the second data set may be generated (e.g., reconstructed) from preacquired second image data.

The time-resolved first data set may map the region of interest of the examination subject in two-dimensional (2D) and/or three-dimensional (3D) spatially resolved form. The examination subject may be a human and/or animal patient, for example. Further, the region of interest may include a spatial region of the examination subject that contains an anatomical object (e.g., an organ, such as a hollow organ), and/or a tumor, and/or a predefined section of the anatomical object. The vessel segments may include an arterial and/or venous vessel segment (e.g., an artery and/or vein) of the examination subject. The tissue regions may, for example, include a brain tissue, muscle tissue, fat tissue, and/or tumor tissue. Further, the first data set may map the region of interest in the first time period, where the first time period includes a plurality of first acquisition time points. The first time period may be, for example, a preprocedural time period. In this case the first data set may map the first contrast agent flow (e.g., a diffusion movement, such as an uptake, and/or flow movement) of a contrast agent (e.g., of a contrast agent bolus) in the region of interest during the first time period. The first data set may have a plurality of first data points, each having a time intensity curve.

The time-resolved second data set may map the region of interest of the examination subject in two-dimensional (2D) and/or three-dimensional (3D) spatially resolved form. The second data set may also map the region of interest in the second time period, where the second time period includes a plurality of second acquisition time points. In this case, the second data set may map the second contrast agent flow (e.g., a diffusion movement, such as an uptake, and/or flow movement) of a contrast agent (e.g., of a contrast agent bolus) in the region of interest during the second time period. The second time period may be, for example, a peri- and/or postprocedural time period (e.g., chronologically following the first time period). The second data set may include a plurality of second data points, each having a time intensity curve.

The time intensity curves may in this case map a change over time in data values of the respective data points (e.g., of the first and second data points). The data values may include, for example, attenuation values, signal values, and/or intensity values.

The spatial registration of the first and the second data set may, for example, include a rigid and/or non-rigid transformation (e.g., a translation and/or rotation and/or scaling and/or deformation) of the first data set and/or the second data set (e.g., of the respective data points). The spatial registration may be based on anatomical and/or geometric features that are mapped in the registered first data set and the registered second data set. The anatomical features may, for example, include an organ (e.g., a hollow organ) and/or an anatomical landmark (e.g., an ostium). Further, the geometric features may, for example, include a contour and/or a marker object. In this case, the spatial registration may include determining the transformation and/or applying the transformation to the first data set and/or the second data set such that a spatial deviation between the mappings in each case of one of the anatomical and/or geometric features in the first data set and the second data set is minimized.

The temporal registration of the first data set and of the second data set may include a synchronization of the time intensity curves of data points of the first data set and the second data set spatially corresponding to one another (e.g., in each case to a data point of the first and the second data set), based on the mapped uptake of the first contrast agent flow and the second contrast agent flow in the at least one vessel segment. In this case, the data points spatially corresponding to one another in the first data set and the second data set, which are used, for example, also for synchronizing the time intensity curves of the remaining data points of the first data set and the second data set, may map a common spatial position in the at least one vessel segment. Further, the temporal difference of the mapped uptake of the first contrast agent flow and the second contrast agent flow in the at least one vessel may be determined by a comparison of time points at which a predefined intensity threshold value is reached or exceeded in the time intensity curves of the data points spatially corresponding to one another in the first data set and the second data set. As a result of the temporal registration of the first data set and the second data set (e.g., the synchronization of the time intensity curves of data points spatially corresponding to one another in the first data set and the second data set), the temporal difference of the mapped uptake of the first contrast agent flow and the second contrast agent flow in the at least one vessel segment may be minimized.

The classification of areas into first areas that include contrasted vessel segments and second areas that include a tissue region may be accomplished by a segmentation of the registered data sets. This segmentation may be based on intensity threshold values, an edge detection, machine learning algorithms, region-shrinking methods, and/or region-growing methods.

The deviation between the second regions of the registered first data set and the registered second data set may be identified by a comparison of the time intensity curves of corresponding data points of the registered first data set and the registered second data set that map the second areas. This comparison may, for example, include a calculation of a difference between the time intensity curves, an analysis of curve parameters of the time intensity curves (e.g., of a maximum intensity), and/or of a time until the maximum intensity is reached.

Further, providing the comparison data set may include a quantitative comparison of the second regions of the registered first and second data set (e.g., a quantitative comparison between the data points of the registered first and second data set that map the deviation between the second areas of the first data set and the second data set). The comparison data set may contain the at least one parameter characterizing the deviation. In this case, the at least one parameter characterizing the deviation may describe (e.g., quantify) the deviation globally (e.g., for the entire region of interest), locally (e.g., data point by data point), or regionally (e.g., for at least one region). Further, the comparison data set may contain multiple parameters characterizing the deviation that describe the deviation locally or regionally. Further, the comparison data set may contain a spatially resolved 2D and/or 3D comparison image of the region of interest of the examination subject, where the data points of the comparison image reflect the deviation between the second areas of the registered first data set and the registered second data set (e.g., a difference or a ratio). The comparison image may also be time-resolved. In this case, data values of the data points of the comparison image may at least partially correspond to the plurality of parameters characterizing the deviation.

Providing the comparison data set may, for example, include storing the data set on a computer-readable storage medium and/or displaying a graphical representation of the comparison data set on a visualization unit and/or transferring the data set to a provisioning unit. For example, providing the comparison data set may include displaying a graphical representation of the comparison data set (e.g., of the at least one parameter characterizing the deviation) using a visualization unit. In addition, providing the comparison data set may include displaying a graphical representation of the registered first data set and the registered second data set (e.g., presented in the form of an overlay or as a side-by-side arrangement). Further, at least one visualization parameter of the visualization unit may be adjusted for displaying the graphical representations as a function of the comparison data set (e.g., of the at least one parameter characterizing the deviation), for example, using color coding.

The comparison data set may also be provided in the form of a map that visualizes the identified deviations. The parameter characterizing the deviation may be a numerical value that quantifies the scale of the deviation and/or a categorical value that describes a type of the deviation.

The method of the present embodiments may offer the advantage that changes in the tissue perfusion between two time points may be efficiently and precisely identified and quantified. This may be particularly useful in order to assess the effects of interventions on the tissue perfusion.

In a further embodiment of the method, identifying the deviation may include extracting a blood flow parameter from the respective time intensity curves.

The extraction of the blood flow parameter may be accomplished, for example, using a computer-implemented analysis of the time intensity curves. This analysis may be based on one or more parameters of the time intensity curves. The computer-implemented analysis may also use sophisticated algorithms such as machine learning or neural networks in order to identify complex patterns in the time intensity curves and derive meaningful blood flow parameters therefrom. Further, the analysis may also include a normalization of the time intensity curves in order to compensate for differences in the contrast agent administration and/or different acquisition conditions.

The extraction of the blood flow parameter may be performed for each data point of the registered first data set and the registered second data set that maps one of the identified second areas.

The extraction of a blood flow parameter from the time intensity curves may enable a quantitative analysis of the contrast agent distribution in the mapped areas of the region of interest. In this case, the blood flow parameter may characterize various aspects of the perfusion, such as, for example, a rate of the blood flow, a blood volume, and/or a perfusion of the tissue.

The extraction of the blood flow parameter may enable subtle changes in the tissue perfusion to be quantified that would possibly not be detectable by visual inspection alone. This may facilitate improved diagnostic support and/or treatment planning in a variety of medical applications.

In a further embodiment of the method, the extraction of the blood flow parameter from the respective time intensity curve may be based on at least one of the following parameters of the respective time intensity curve: an area under the time intensity curve, a maximum intensity, and/or a length of time from an increase in intensity up to a maximum intensity.

The extraction of the blood flow parameter may be based on different characteristics of the time intensity curve for the purpose of obtaining information about the blood flow. The area under the time intensity curve may be a metric for a total volume of the contrast agent that has passed through a particular region. A larger area under the time intensity curve may be indicative of a stronger blood flow. The maximum intensity may reflect the highest concentration of the contrast agent in the area under consideration. A higher maximum value may indicate a better perfusion. The length of time from an increase in intensity up to a maximum intensity may give an indication of the rate of the blood flow. A shorter time period may indicate a faster blood flow. Further, the length of time from an increase in intensity up to a maximum intensity may be used for determining a wash-in rate of the contrast agent.

Further, a combination of these parameters may be used in order to obtain a more comprehensive picture of the blood flow. For example, the area under the time intensity curve may be considered in conjunction with the maximum intensity in order to take into account both the total volume and the highest concentration of the contrast agent.

Using these parameters for extracting the blood flow parameter enables a quantitative analysis of blood flow changes in the region of interest of the examination subject to be conducted. This may be particularly useful in order to identify subtle changes in blood flow.

In a further embodiment of the method, identifying the deviation may include identifying an area-by-area blood flow change based on the extracted blood flow parameters, where the comparison data set may be provided in a manner in which it has at least one parameter characterizing the area-by-area blood flow change.

An area-by-area blood flow change may be identified using a comparison of the extracted blood flow parameters for corresponding (e.g., spatially and/or functionally corresponding) areas in the registered first data and the registered second data set. The areas may be determined in different ways (e.g., based on anatomical regions, functional units, and/or based on a segmentation of the registered first data set and the registered second data set). In this case, the areas may also be defined manually by a user (e.g., using annotation). A blood flow change may be identified, for example, as a (e.g., percentage) deviation between the blood flow parameters of the registered first data set and the registered second data set.

The comparison data set may have at least one parameter for characterizing the area-by-area blood flow change. The at least one parameter may include an absolute or relative change in the blood flow parameters (e.g., a direction of the change, such as an increase or decrease) and/or a classification of the change (e.g., major, moderate, or minor). In addition, spatial information such as a size and/or shape of the regions with changed blood flow may be included in the comparison data set. Further, temporal information, such as a duration and/or a variation with time of the blood flow change, may also be included in the comparison data set.

Providing the comparison data set containing parameters for characterizing the area-by-area blood flow change may enable a quantitative analysis of the perfusion changes. This may be useful in order to judge a success of an intervention or to identify unintended effects on the tissue perfusion. The parameters may also serve as a basis for an automated assessment or classification of the perfusion changes. Further, the comparison data set may also be used to identify trends or patterns in the perfusion changes over greater areas or longer time periods.

In a further embodiment of the method, the classification of the areas may include a vessel and/or tissue segmentation in the registered first data set and the registered second data set.

The vessel and/or tissue segmentation may be performed in different ways in order to identify areas with and without vessel segments in the first data set and the second data sets. During the vessel and/or tissue segmentation, data points of the registered first data set and the registered second data set (e.g., pixels or voxels) may be classified based on their respective intensity values as data points mapping a vessel segment or tissue region. The vessel and/or tissue segmentation may be based on threshold values. For example, the vessel and/or tissue segmentation may include applying a band-rejection filter followed by a threshold value comparison. Further, algorithms based on active contours, level-set methods, and/or machine learning may be used for vessel and/or tissue segmentation.

The vessel segmentation may facilitate an identification of vessel segments mapped in the registered first data set and the registered second data set. In this case, anatomical and/or geometric features of the vessel segments (e.g., a tube-shaped structure of the vessel segments), a contrasting using the contrast agent arranged therein, and/or branching patterns may be used for the vessel segmentation.

The tissue segmentation may facilitate an identification of tissue regions mapped in the registered first data set and the registered second data set. The tissue regions may include, for example, a brain tissue, muscle tissue, fat tissue, and/or tumor tissue. For example, areas of the region of interest that include substantially no (e.g., contrasted) vessel segments may be classified as a tissue region. The segmentation of the tissue regions may be based on intensity values, texture features, and/or anatomical and/or geometric features of the tissue regions. In some cases, functional tissue properties (e.g., a contrast agent uptake) may also be referred to for the classification.

Further, a combined vessel and tissue segmentation may be performed in which both the vessel segments and tissue regions are segmented. This may allow a more accurate distinction between vessel segments and tissue regions.

The segmentation may be performed separately for the first data set and the second data set. Alternatively, the segmentation of the first data set may serve as a starting point for a segmentation of the second data set or vice versa.

Areas that include contrasted vessel segments or tissue regions may be identified by the vessel and/or tissue segmentation. These areas may then be used for the further analysis of the perfusion changes since they represent the surrounding tissue in which changes in the blood flow may be of special interest. Conducting a vessel and/or tissue segmentation may facilitate a more precise analysis of the perfusion changes since the analysis may be concentrated on the relevant tissue regions and potential perturbations (e.g., caused by large vessel segments) may be minimized. This may lead to an improved detection and quantification of perfusion changes, which may be advantageous for the clinical assessment and decision making during interventional methods.

In a further embodiment of the method, at least one proximal area of the region of interest that is mapped in the registered first data set and the registered second data set may be identified based on the vessel segmentation and the area by area blood flow change, where the time intensity curves of the proximal area may have a comparatively shortest time up to a maximum intensity in the vessel segmentation in an area exhibiting comparatively reduced blood flow. The comparison data set containing an identification of the at least one proximal area may be provided.

The identification of the proximal area may be based on a combination of the vessel segmentation and the determined area by area blood flow change. In this case, the vessel segmentation may be performed initially in order to identify respective images of the vessel segments in the registered first data set and the registered second data set. The area-by-area blood flow change may then be analyzed in order to localize regions with reduced blood flow. The proximal area may be identified (e.g., determined) among the identified second areas that include no vessel segment.

In the identified region with reduced blood flow, the time intensity curves of the segmented second areas may then be examined. The proximal area may be identified as at least one subregion of one of the second regions in which the time intensity curves take the shortest time to reach the maximum intensity. This may indicate that it is the subregion that is located closest to the contrast agent source of the second region in which a change in the blood flow occurs.

The comparison data set having the identification of the proximal area may be provided in various forms. For example, a graphical highlighting of the proximal area may be used in an overlay representation of the second areas of the registered first data set and the registered second data set. Alternatively or in addition, the comparison data set may include numerical coordinates or anatomical descriptions of the proximal area.

The identification of the proximal area may provide a user (e.g., a member of the medical staff) with important information (e.g., information relating to an origin of a vascular occlusion or stenosis). This method for identifying and providing the proximal area may increase the degree of efficiency and accuracy in the analysis of changes in the second regions of the registered first and second data set. The embodiment may support a targeted examination and treatment of pathologies in the second areas and thus contribute to improved clinical results.

In a further embodiment of the method, the spatial and temporal registration of the first data set and the second data set may include a normalization of the time intensity curves of the first data set and the second data set.

The normalization of the time intensity curves may serve to compensate for differences in the acquisition conditions between the first data set and the second data set and to support an improved comparability of the data sets. In this case, the data values (e.g., the time intensity curves) of the data points of the first data set and the second data set may be normalized to a data value of the data point having a highest intensity. The data point having the highest intensity may be localized at a point of the contrast agent inflow (e.g., may map the point of the contrast agent inflow).

The normalization may be performed in various ways. One possibility may be to divide the intensity values of the data points by the maximum intensity value in the respective data set. Alternatively, the intensity values may be scaled to a specific value range (e.g., between 0 and 1).

Differences in the contrast agent volume or concentration that may occur between the acquisitions of the first data set and the second data set may be compensated for using the normalization. This may lead to an improved accuracy in the identification of deviations between the data sets.

The normalized time intensity curves may subsequently be used for the further processing and analysis (e.g., for the comparison between the registered first data set and the registered second data set in order to identify deviations between the second areas of the registered first and the registered second data set, such as to identify perfusion changes in the tissue regions of the examination subject).

In a further embodiment of the method, the identification of the deviation may include a data point by data point identification of a respective deviation between the first and second data points of the registered first data set and the registered second data set.

A respective deviation may be identified (e.g., determined) for (e.g., spatially) corresponding first and second data points. This may facilitate a very detailed and precise analysis of changes between the two acquisition time points.

The identification, data point by data point, may be performed, for example, using a direct comparison of the time intensity curves and/or of the blood flow parameters extracted therefrom for the corresponding first and second data points. In this case, different comparison metrics such as absolute or relative differences, ratios, and/or statistical measures may be used.

An advantage of this embodiment may be that small-scale or local changes in perfusion may also be registered, which would possibly be overlooked in a coarser analysis. Further, the analysis, data point by data point, may form a basis for more detailed spatial evaluations or visualizations of the perfusion changes.

In a further embodiment of the method, providing the comparison data set may include providing a graphical representation based on the deviation using a visualization unit.

Providing the comparison data set may include providing (e.g., displaying) a graphical representation of the comparison data set (e.g., of the at least one parameter characterizing the deviation) using a visualization unit. In addition, providing the graphical representation may include displaying (e.g., in the form of an overlay or as a side-by-side arrangement) a graphical representation of the registered first and second data set. Further, at least one visualization parameter of the visualization unit may be adjusted for displaying the graphical representation as a function of the comparison data set (e.g., of the at least one parameter characterizing the deviation, such as using color coding).

The graphical representation may be realized in different ways in order to visualize the identified deviations between the registered first data set and the registered second data set. In this case, regions having increased or reduced tissue perfusion may be highlighted. A threshold value may be used for this purpose (e.g., a percentage deviation of 20% between the extracted blood flow parameters of the registered first data set and the registered second data set).

The visualization unit may include a screen, a monitor, or another suitable display device. The graphical representation may be color-coded, where different colors may represent different degrees of the deviation. For example, regions having increased perfusion may be represented in shades of red and regions having reduced perfusion in shades of blue.

Further, the graphical representation of the deviation may be superimposed as an overlay on a vessel representation of the examination subject. This may allow the user to consider the perfusion changes in the context of the anatomical structures. The vessel representation may be extracted from the first data set or second data set or be sourced from a separate acquisition.

The graphical representation may be configured as interactive such that the user may choose different views, zoom in and out of the visualization, or mark particular areas. This may facilitate an analysis and interpretation of the perfusion changes.

By providing such a graphical representation, an intuitive and efficient visualization of the perfusion changes may be realized. This may help the user to quickly and precisely identify areas having significant changes and to make appropriate clinical decisions.

In a further embodiment of the method, the deviation may be compared (e.g., data point by data point) with at least one predefined threshold value. The deviation (e.g., data point by data point) may be masked out of the graphical representation as a function of the comparison (e.g., if the predefined threshold value is undershot or exceeded).

The comparison of the deviation with the predefined threshold value may enable, for example, significant changes to be identified. The deviation may be characterized by a deviation value, such as, for example, a difference between the time intensity curves or extracted blood flow parameters of corresponding second regions of the registered first and the registered second data set. The threshold value may be defined, for example, as a percentage value or as an absolute value. The threshold value may be able to be altered by the user in order to adjust a sensitivity of the graphical representation. The comparison may be conducted data point by data point or for defined areas.

According to a first variant, deviations below the predefined threshold value may be masked. At the same time, deviations that exceed the predefined threshold value or are equal to the predefined threshold value may be highlighted in the graphical representation. The masking of the deviation out of the graphical representation if the threshold value is undershot may allow a more clearly interpretable visualization. Areas with minor changes that are possibly due to noise or natural fluctuations may be masked out.

According to a second variant, deviations above the predefined threshold value may be masked. At the same time, deviations that undershoot the predefined threshold value or are equal to the predefined threshold value may be highlighted in the graphical representation. The masking of the deviation out of the graphical representation if the threshold value is exceeded may allow a more clearly interpretable visualization. Areas with comparatively large changes may be masked out.

This may help the user to concentrate on the relevant changes. The masking of the deviation out of the graphical representation as a function of the comparison (e.g., if the predefined threshold value is undershot or exceeded) may include an at least partial (e.g., complete) masking out of the corresponding data points and/or areas in the graphical representation. Alternatively or in addition, a graduated graphical representation may be used in which an intensity, transparency, and/or coloration of the graphical representation correlates with a degree of the deviation.

Performing the comparison data point by data point together with the masking may enable precise control over the representation. Each data point may be evaluated individually and handled as appropriate in the graphical representation. This may lead to a detailed and meaningful visualization of the perfusion changes.

The combination of threshold value comparison and masking may improve an interpretability of the results and support clinical decision making. Physicians may concentrate on the areas having the most significant changes, which may increase the efficiency of the analysis.

In a further embodiment of the method, providing the comparison data set may include providing a graphical representation based on an at least partial overlaying of the deviation with a vessel representation of the examination subject.

The graphical representation may allow a visual representation of the identified deviations in relation to the anatomical structure of the examination subject. In this regard, the vessel representation may serve as a reference in order to visualize a spatial distribution of the deviations in the context of the vascular anatomy.

The vessel representation may include a spatially resolved (e.g., 2D or 3D) representation of a vessel structure (e.g., of one or more vessel segments) of the examination subject (e.g., of the region of interest). The vessel representation may also be time-resolved. The vessel representation may include a mapping (e.g., medical image data and/or a reconstruction based on medical image data) and/or a model (e.g., a centerline model and/or a volume net model) of the vessel structure of the region of interest. The vessel representation may be derived based on the first data set, the second data set, or a combination of both data sets (e.g., the first areas of the first data set and/or the second data set).

Alternatively, the vessel representation may have been determined based on further medical image data of the examination subject. For example, the vessel representation may have been generated using a reconstruction from a plurality of 2D acquisitions or directly from a 3D imaging method such as computed tomography angiography (CTA) or magnetic resonance angiography (MRA). Further, the vessel representation may also include a parametric map. In this case, different vessel properties such as diameter, flow rate, or wall condition may be represented (e.g., using color coding).

The vessel representation may be registered with the deviation (e.g., with the registered first data set and the registered second data set).

The overlay may include an at least partial (e.g., complete) overlaying (e.g., projection) of the deviation (e.g., of the at least one parameter characterizing the deviation) onto the vessel representation. Alternatively, a partly transparent layer containing the deviation information may be placed over the vessel representation.

This type of visualization may enable the user to quickly and intuitively identify regions (e.g., tissue regions) exhibiting significant changes in blood flow and to register their spatial relationship to the vessel structures. This may be particularly useful in order to assess the effects of an intervention on the tissue perfusion or to localize regions with abnormal blood flow.

The overlay may be dynamically adjustable such that the user may vary the transparency or intensity of the deviation representation in relation to the vessel representation. This may increase a flexibility in an analysis of the graphical representation and allow a detailed examination of specific regions of interest.

In a further embodiment of the method, providing the comparison data set may include providing an identification of vessels that are mapped in the first data set and not in the second data set and/or that are mapped in the second data set and not in the first data set.

Identifying the vessels that are mapped in the first data set and not in the second data set may include a comparison of the first areas of the first data set with the first areas of the second data set. In this case, first data points that map the vessels (e.g., vessel segments) may be identified, while corresponding second data points do not map any vessels (e.g., vessel segments). Analogously thereto, the identification of the vessels that are mapped in the second data set and not in the first data set may include a comparison of the first areas of the second data set with the first areas of the first data set. In this case, second data points may be identified that map the vessels (e.g., vessel segments), while corresponding first data points do not map any vessels (e.g., vessel segments).

Providing the identification of the vessels that are mapped in the first data set and not in the second data set and/or that are mapped in the second data set and not in the first data set may include different visualization and/or annotation techniques. For example, a representation (e.g., a mapping and/or a model) of the identified vessels (e.g., vessel segments) may be highlighted in the graphical representation of the comparison data set. The highlighting of the representation of the identified vessels may include, for example, a colored highlighting, a color coding, a contouring, and/or a marking of borders of the identified vessels. Providing the identification of the vessels may further include providing an annotation of the identified vessels. The annotation of the vessels may include, for example, text, symbols, and/or numbers in order to indicate the identified vessels. These annotations may be configured as interactive so that the user may retrieve additional information using a mouse click or by touching the graphical representation.

Providing the identification of the vessels may further include providing positional information. The positional information may include coordinates, anatomical reference points, and/or a description of a location relative to known structures (e.g., anatomical landmarks). Such information may be displayed in a separate text field or as part of the graphical representation.

Providing the identification of the vessels may also be realized by a selective transparency. This may make it possible for the user to register the spatial distribution of the identified vessels in the context of the overall vessel structure.

This functionality may be useful in order to visualize changes in the vessel structure between the two acquisition time points. It may provide pointers to vascular occlusions, embolisms, or other changes in the blood supply that may have occurred between the acquisition time points. The visualization of this information may help the user to identify and assess changes in the vessel structure.

The present embodiments relate, in a second aspect, to a provisioning unit that is configured for performing a proposed method for providing a comparison data set.

The advantages of the provisioning unit of the present embodiments substantially correspond to the advantages of the method for providing a comparison data set of the present embodiments. Features, advantages, or alternative embodiments mentioned in this context may equally be applied also to the other subject matters and vice versa.

The provisioning unit may include a computing unit (e.g., including one or more processors), a memory unit (e.g., including memory) and/or an interface. The provisioning unit (e.g., the components of the provisioning unit) may be configured for performing the individual acts of the method of the present embodiments for providing a comparison data set. The interface may be configured for providing the time-resolved first data set and the second data set and for providing the comparison data set. The computing unit and/or the memory unit may be configured for the spatial and temporal registration of the first data set and the second data set, for classifying areas of the region of interest that are mapped in the registered first data set and the registered second data set, for identifying a deviation between the second regions of the registered first data set and the registered second data set, as well as for providing the comparison data set.

The present embodiments relate, in a third aspect, to a medical imaging device that includes a proposed provisioning unit. The medical imaging device may be configured to acquire and/or provide the first data set and second data set.

The medical imaging device may be configured, for example, as a magnetic resonance tomography system (MRT) and/or computed tomography system (CT) and/or medical X-ray machine and/or positron emission tomography system (PET) and/or ultrasound device. Further, the medical imaging device may be configured to acquire the first data set and the second data set of the examination subject including the region of interest. Alternatively, the medical imaging device may be configured to acquire first and second image data (e.g., first and second projection mappings) of the examination subject including the region of interest. In this case, the medical imaging device may be further configured to reconstruct and provide the first data set from the first image data. The medical imaging device may be further configured to reconstruct and provide the second data set from the second image data.

The advantages of the medical imaging device of the present embodiments substantially correspond to the advantages of the method for providing a comparison data set and/or the provisioning unit of the present embodiments. Features, advantages, or alternative embodiments mentioned in this context may equally be applied also to the other subject matters and vice versa.

The present embodiments relate, in a fourth aspect, to a computer program product including a computer program that may be loaded directly into the memory of a provisioning unit. The computer program includes program sections for performing all the acts of a method of the present embodiments for providing a comparison data set when the program sections are executed by the provisioning unit.

The advantages of the computer program product of the present embodiments substantially correspond to the advantages of the method for providing a comparison data set of the present embodiments. Features, advantages, or alternative embodiments mentioned in this context may equally be applied also to the other subject matters and vice versa.

The computer program product may, for example, include software having a source code that still needs to be compiled and linked or that only needs to be interpreted, or an executable software code that still has to be loaded into the provisioning unit in order to execute. The computer program product enables the method for providing a comparison data set to be performed quickly, identically reproducibly, and robustly using a provisioning unit. The computer program product is configured such that the computer program product may perform the method acts according to the present embodiments using the provisioning unit.

The computer program product is stored, for example, on a computer-readable storage medium or held resident on a network or server, from where it may be loaded into the processor of a provisioning unit that is connected directly to the provisioning unit or may be configured as part of the provisioning unit. Further, control information of the computer program product may be stored on an electronically readable data medium (e.g., a non-transitory computer-readable storage medium). The control information of the electronically readable data medium may be configured such that the control information performs a method according to the present embodiments when the data medium is used in a provisioning unit. Examples of electronically readable data media include a DVD, a magnetic tape, or a USB stick on which electronically readable control information (e.g., software) is stored. When this control information is read from the data medium and stored in a provisioning unit, all the embodiments of the above-described methods may be performed.

A largely software-based implementation has the advantage that provisioning units already used previously may also be easily upgraded by a software update in order to operate in the manner according to the present embodiments. In addition to the computer program, such a computer program product may, where appropriate, include additional constituent parts, such as, for example, a set of documentation, and/or additional components, including hardware components, such as, for example, hardware keys (e.g., dongles, etc.), to enable use of the software.

### BRIEF DESCRIPTION OF THE DRAWINGS

Example embodiments of the invention are illustrated in the drawings and are explained in more detail below. Like reference characters are used for like features in different figures, in which:

FIGS. 1 and 2 show schematic representations of embodiments of a method for providing a comparison data set;

FIG. 3 shows a schematic view of a preprocedural contrast-enhanced vessel image;

FIG. 4 shows a schematic view of a postprocedural contrast-enhanced vessel image;

FIG. 5 shows a schematic view of a preprocedural contrast-enhanced tissue image;

FIG. 6 shows a schematic view of a postprocedural contrast-enhanced tissue image;

FIG. 7 shows a diagram containing preprocedural time intensity curves;

FIG. 8 shows a diagram containing postprocedural time intensity curves;

FIG. 9 shows a schematic view of an example overlay image;

FIG. 10 shows a schematic representation of an embodiment of a provisioning unit; and

FIG. 11 shows a schematic view of an embodiment of a medical C-arm X-ray machine.

### DETAILED DESCRIPTION

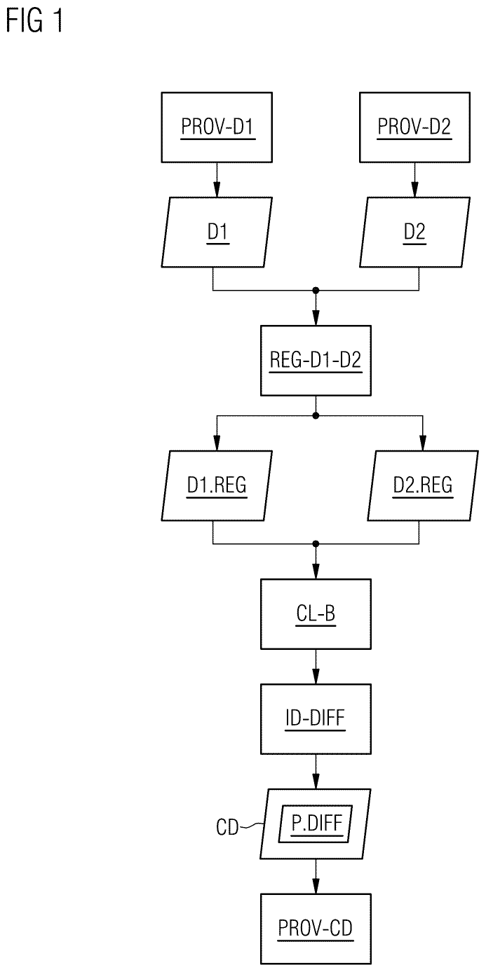

FIG. 1 shows a schematic representation of an embodiment of a method for providing a comparison data set. The method may include a providing PROV-D1 of a time-resolved first data set D1. The first data set D1 may include first data points, each having a time intensity curve. The time intensity curves of the first data points may map a first contrast agent flow in a region of interest IR of an examination subject in a first time period. The method may further include a providing PROV-D2 of a time-resolved second data set D2. The second data set D2 may include second data points, each having a time intensity curve. The time intensity curves of the second data points may map a second contrast agent flow in the region of interest IR of the examination subject in a second time period. The method may include a spatial and temporal registration REG-D1-D2 of the first data set D1 and the second data set D2. This enables a registered first data set D1.REG and a registered second data set D2.REG to be provided. The method may include a classification CL-B of areas of the region of interest that are mapped in the registered first data set D1.REG and the registered second data set D2.REG. The areas may be classified into first areas that include contrasted vessel segments, and second areas that include a tissue region. The method may include an identifying ID-DIFF of a deviation between the second areas of the registered first data set D1.REG and the registered second data set D2.REG. The identification ID-DIFF may be based on a comparison of the time intensity curves of the registered first data set D1.REG and the registered second data set D2.REG that map the second areas. The method may include a providing PROV-CD of the comparison data set CD based on the registered first data set D1.REG and the registered second data set D2.REG. The comparison data set CD may contain at least one parameter characterizing the deviation P.DIFF.

The identification ID-DIFF of the deviation may include an identification, data point by data point, of a respective deviation between the first data points and the second data points of the registered first dataset D1.REG and the registered second data set D2.REG, respectively.

The provision of the comparison data set PROV-CD may include a providing of a graphical representation based on the deviation using a visualization unit. The deviation may be compared data point by data point with a predefined threshold value. The data point by data point deviation may be masked out of the graphical representation as a function of the comparison (e.g., if the predefined threshold value is undershot or exceeded).

The provision of the comparison data set CD may, for example, include a providing of a graphical representation based on an at least partial overlaying of the deviation with a vessel representation of the examination subject.

The providing PROV-CD of the comparison data set CD may further include a providing of an identification of vessels P.V that are mapped in the first data set D1 and not in the second data set D2 and/or that are mapped in the second data set D2 and not in the first data set D1.

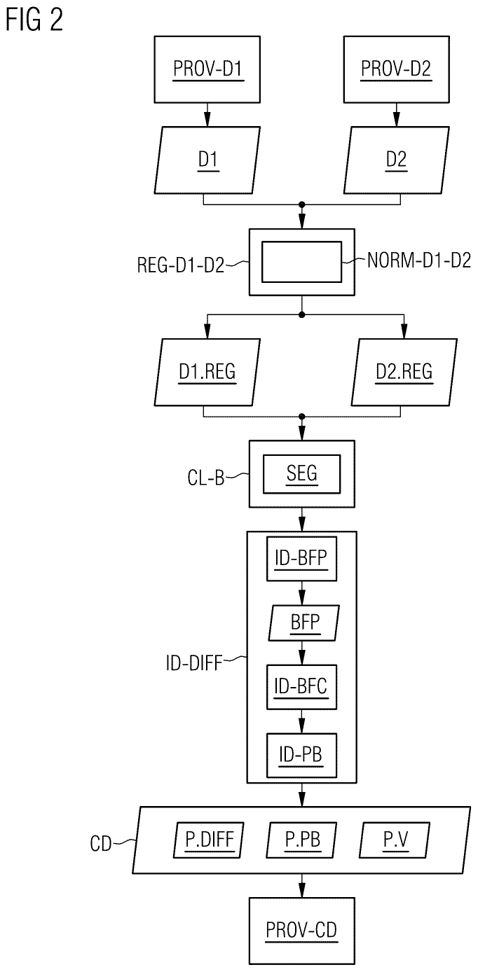

FIG. 2 shows a schematic representation of a further embodiment of a method for providing a comparison data set. The spatial and temporal registration REG-D1-D2 of the first data set D1 and the second data set D2 may include a normalization NORM-D1-D2 of the time intensity curves of the first data set D1 and the second data set D2. The method may include an extraction ID-BFP of a blood flow parameter BFP from the respective time intensity curves of the first data set D1 and the second data set D2. The extraction ID-BFP of the blood flow parameter BFP from the respective time intensity curve may be based on at least one of the following parameters of the respective time intensity curve: an area under the curve, a maximum intensity, and a length of time from an increase in the intensity up to a maximum intensity.

The area under the curve (AUC) may be calculated using the following formula:

$$AUC = {\int }_{t 1}^{t 2} I ( t ) dt \qquad ( 1 )$$

where I (t) is the intensity at time point t, t1 is the start time point, and t2 is the end time point of the measurement. This calculation may be approximated using respective intensity values Iᵢ of the time intensity curves for n discrete acquisition time points tᵢ, where i denotes an index of 1 to n. This calculation may yield an approximation of the area under the curve and thus contribute to the quantification of the blood flow.

The identifying ID-DIFF of the deviation may include a determination, data point by data point, of pre-post changes. The change in the area under the curve (Δ AUC) between the pre- and postprocedural state may be calculated as Δ AUC=AUC_post−AUC_pre. This calculation may allow a direct comparison of the tissue perfusion before and after the procedure. A positive Δ AUC value may indicate an increase in the perfusion, whereas a negative value may indicate a decrease. The magnitude of the Δ AUC value may represent the extent of the change in perfusion.

The calculation of AUC_post and AUC_pre may be performed, for example, as described in equation (1), where t1 and t2 are predefined for the respective time periods (e.g., the first time period and the second time period). The calculation may be carried out for each data point in the respective data sets. Different numerical integration methods such as the trapezoidal rule or Simpson's rule may be used in this process in order to approximate the area under the discrete measurement points. In some cases, a curve fitting to the measurement points may also be performed in order to obtain a continuous function for the integration. The choice of integration method may be dependent on factors such as the number of measurement points, the shape of the curve, and the desired accuracy.

Further, the length of time from an increase in the intensity up to a maximum intensity may be used for determining a wash-in rate W of the contrast agent:

$$W = \frac{{I}_{max} - {I}_{min}}{TTP} \qquad ( 2 )$$

where Iₘₐₓ denotes the maximum intensity, Iₘᵢₙ denotes a minimum intensity, and TTP denotes the length of time of the increase in the intensity from the minimum intensity Iₘᵢₙ up to the maximum intensity Iₘₐₓ.

The identification of a deviation ID-DIFF between the second areas of the registered first data set and the registered second data set may include an identifying ID-BFC of an area-by-area blood flow change BFC based on the extracted blood flow parameters BFP. The comparison data set CD may be provided PROV-CD having at least one parameter P.DIFF characterizing the area-by-area blood flow change BFC.

The classification of the regions CL-B may include a vessel and/or tissue segmentation SEG in the registered first data set D1.REG and the registered second data set D2.REG.

The vessel and/or tissue segmentation SEG may enable first areas including contrasted vessel segments to be differentiated from second areas including a tissue region. The tissue segmentation may be used to obtain a further classification of the second areas.

Step ID-BFC may include the identification of the area-by-area blood flow change BFC for the second areas that do not contain a vessel segment. In this way, an analysis of the tissue perfusion may be conducted without vessel structures distorting the result.

At least one proximal area of the region of interest IR mapped in the registered first data set D1.REG and the registered second data set D2.REG may be identified ID-PB based on the vessel segmentation SEG and the area-by-area blood flow change BFC. The time intensity curves of the proximal area may exhibit a comparatively shortest time up to a maximum intensity in the vessel segmentation SEG in an area exhibiting comparatively reduced blood flow. The comparison data set CD may be provided having an identification P.PB of the at least one proximal area.

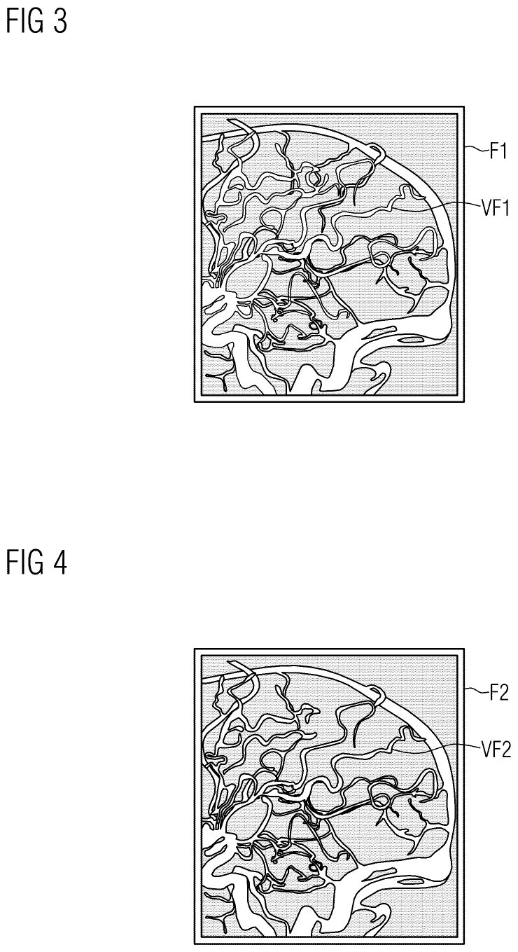

FIG. 3 shows a schematic view of a preprocedural contrast-enhanced vessel image F1. The preprocedural contrast-enhanced vessel image F1 may visualize the region of interest (e.g., a brain region) of the examination subject in the first time period. Vessel segments VF1 preprocedurally filled with contrast agent may be visible against a tissue background.

FIG. 4 shows a schematic view of a postprocedural contrast-enhanced vessel image F2. The postprocedural contrast-enhanced vessel image F2 may visualize the region of interest of the examination subject 31 in the second time period. Vessel segments VF2 postprocedurally filled with contrast agent may be visible against a tissue background.

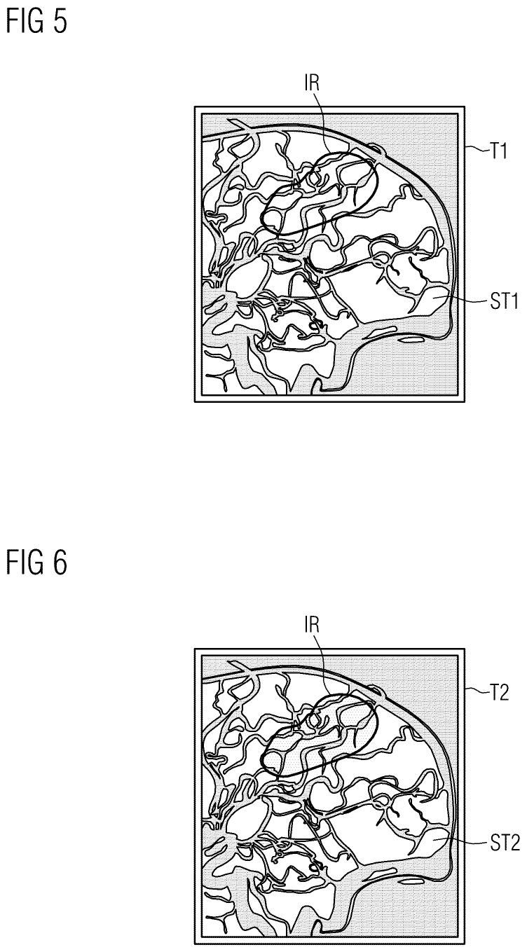

FIG. 5 shows a schematic view of a preprocedural contrast-enhanced tissue image T1. The preprocedural contrast-enhanced tissue image T1 may visualize the region of interest IR of the examination subject 31 in the first time period. The preprocedural contrast-enhanced tissue image T1 may visualize tissue regions of the examination subject preprocedurally (e.g., without the vessel segments).

FIG. 6 shows a schematic view of a postprocedural contrast-enhanced tissue image T2. The postprocedural contrast-enhanced tissue image T2 may visualize the region of interest IR of the examination subject 31 in the second time period. The postprocedural contrast-enhanced tissue image T2 may visualize tissue regions of the examination subject postprocedurally (e.g., without the vessel segments).

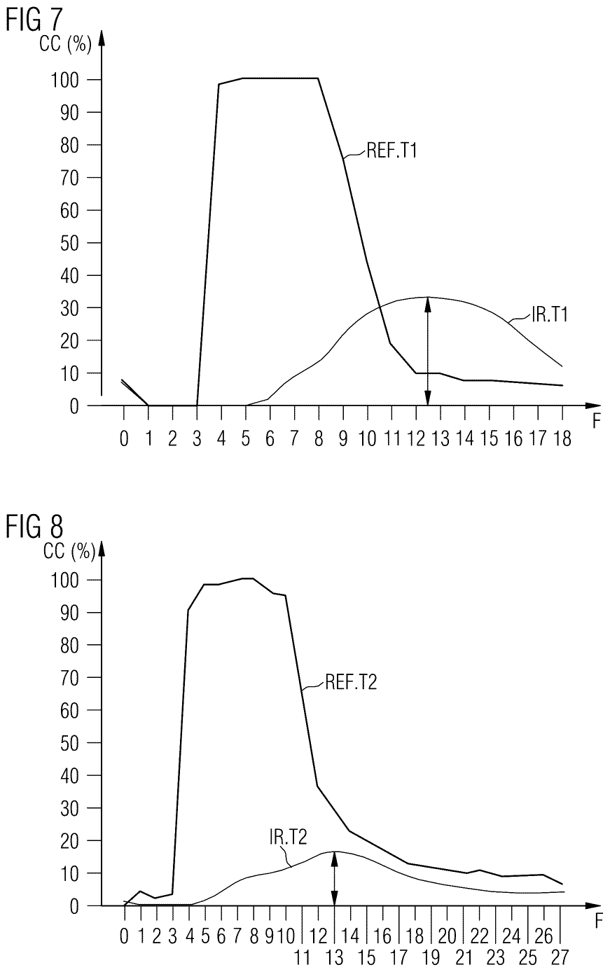

FIG. 7 shows a diagram containing preprocedural time intensity curves. The contrast agent concentration CC in percent (%) is shown on the y-axis, and a number of frames F (e.g., individual images) are shown on the x-axis. The diagram contains two time intensity curves REF.T1 and IR.T1. The time intensity curve REF.T1 represents a preprocedural reference range. The time intensity curve REF.T1 exhibits a rapid rise to a peak value of approximately 100% contrast agent concentration, followed by a steep decline and then a gradual decrease. The time intensity curve IR.T1 represents the preprocedural region of interest IR. The time intensity curve IR.T1 may have a smaller peak value than the time intensity curve REF.T1 that occurs later in time than the peak value of the time intensity curve REF.T1.

FIG. 8 shows a diagram containing postprocedural time intensity curves. The contrast agent concentration CC in percent (%) is shown on the y-axis, and a number of frames F (e.g., individual images) are shown on the x-axis. The diagram contains two time intensity curves REF.T2 and IR.T2. The time intensity curve REF.T2 represents a postprocedural reference range. The time intensity curve REF.T2 exhibits a rapid rise to a peak value of approximately 100% contrast agent concentration, followed by a steep decline and then a gradual decrease. The time intensity curve IR.T2 represents the postprocedural region of interest IR. During all of the measured frames, the time intensity curve IR.T2 remains at a comparatively lower contrast agent concentration level. The time intensity curve IR.T2 may have a smaller peak value than the time intensity curve REF.T2. The smaller peak value occurs later than the peak value of the time intensity curve REF.T2. In comparison with the peak value of the time intensity curve IR.T1, the peak value of the time intensity curve IR.T2 is smaller, which may be indicative of a postprocedurally reduced blood flow in the region of interest IR.

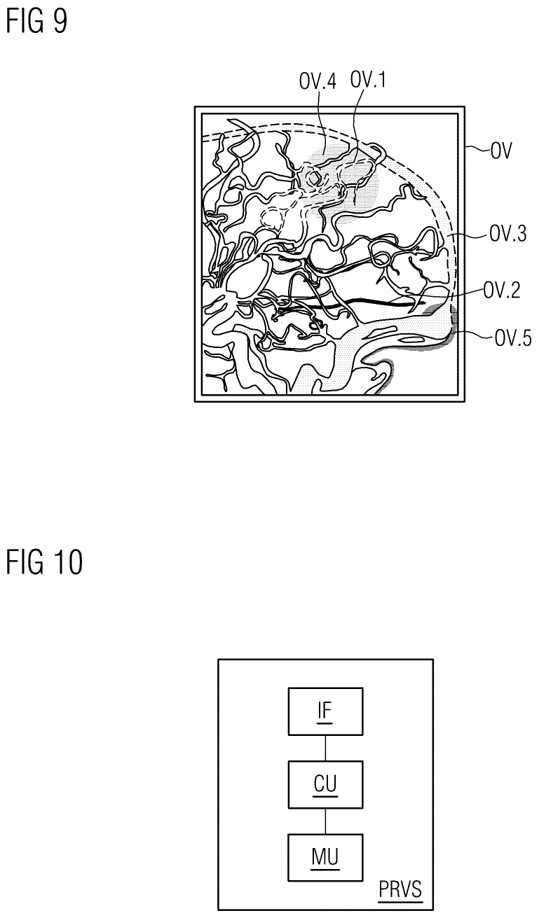

FIG. 9 shows a schematic view of an example overlay image OV. The overlay image OV may include a plurality of elements that visually represent a comparison between pre- and postprocedural vessel structures and tissue perfusion changes.

The provision PROV-CD of the comparison data set CD may include a providing of a graphical representation based on an at least partial overlaying of the deviation with a vessel representation of the examination subject. The overlay image OV may include the vessels OV.1 identified only preprocedurally, the vessels OV.2 identified only postprocedurally, the vessels OV.3 identified pre- and postprocedurally, regions exhibiting postprocedurally reduced tissue perfusion OV.4, and regions exhibiting postprocedurally increased tissue perfusion OV.5.

The overlay image OV may enable a comparison between pre- and postprocedural states of the region of interest IR and highlight regions with changed blood flow and changed vessel structure.

FIG. 10 shows a schematic representation of an embodiment of a provisioning unit PRVS. The provisioning unit PRVS may include a computing unit CU, a memory unit MU, and/or an interface IF. The provisioning unit PRVS (e.g., the components of the provisioning unit PRVS) may be configured for performing the individual acts of the method of the present embodiments for providing a comparison data set. The interface IF may be configured for providing the time-resolved first data set D1 and the time-resolved second data set D2, and for providing the comparison data set CD. The computing unit CU and/or the memory unit MU may be configured for the spatial and temporal registration REG-D1-D2 of the first data set D1 and the second data set D2, for classifying CL-B areas of the region of interest IR mapped in the registered first data set D1.REG and the registered second data set D2.REG, for identifying ID-DIFF a deviation between the second regions of the registered first data set D1.REG and the registered second data set D2.REG, as well as for providing PROV-CD the comparison data set CD.

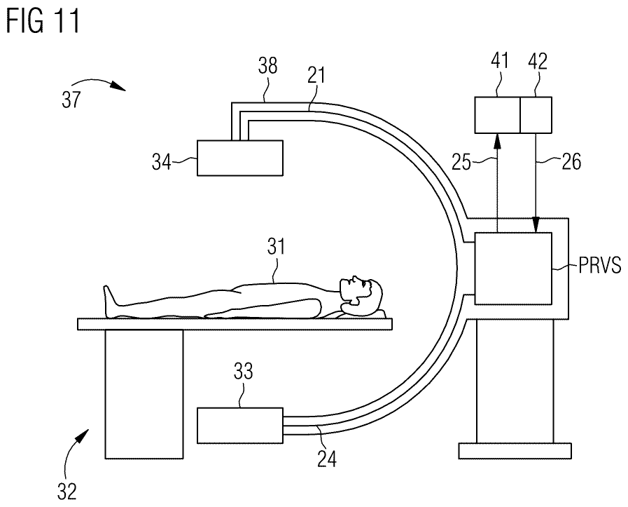

FIG. 11 shows a schematic view of a medical C-arm X-ray machine 37 as an example of a proposed medical imaging device. In this example, the medical C-arm X-ray machine 37 may include a detector 34 (e.g., an X-ray detector) and an X-ray source 33. The medical C-arm X-ray machine 37 may be configured for acquiring and/or for providing the first data set D1 and the second data set D2. For example, the medical C-arm X-ray machine may be configured for acquiring the first image data ID1 including a plurality of first projection images of the region of interest IR of the examination subject 31 and for acquiring the second image data ID2 including a plurality of second projection images of the region of interest IR of the examination subject 31.

In order to acquire the first and second projection images, an arm 38 of the C-arm X-ray machine 37 may be mounted so as to be movable around one or more axes. Also, in order to acquire the first and the second projection images of the examination subject 31 arranged on a patient support and positioning device 32, the provisioning unit PRVS may send a signal 24 to the X-ray source 33. The X-ray source 33 may thereupon emit an X-ray beam. When the X-ray beam, following an interaction with the examination subject 31, impinges on a surface of the detector 34, the detector 34 may send a signal 21 to the provisioning unit PRVS. The provisioning unit PRVS may receive the first and the second projection images based on the signal 21, for example.

In addition, the C-arm X-ray machine 37 may include an input unit 42 (e.g., a keyboard) and the visualization unit 41 (e.g., a monitor and/or display). The input unit 42 may be integrated into the visualization unit 41 (e.g., in the case of a capacitive and/or resistive input display). In this case, it is possible, using an input by a user at the input unit 42, to enable control of the medical C-arm X-ray machine 37 (e.g., of the proposed method for providing PROV-CD a comparison data set CD). The input unit 42 may, for example, send a signal 26 to the provisioning unit PRVS for this purpose.

The visualization unit 41 may be configured for displaying, for example, in the form of an overlay or in a side-by-side arrangement, a graphical representation of the registered first data set D1.REG and/or the registered second data set D2.REG and/or of the comparison data set CD (e.g., of the at least one parameter P.DIFF characterizing the deviation). For this purpose, the provisioning unit PRVS may send a signal 25 to the visualization unit 41. Further, at least one visualization parameter of the visualization unit 41 for displaying the graphical representations may be adjusted as a function of the comparison data set CD (e.g., of the at least one parameter P.DIFF characterizing the deviation, such as using color coding).

The schematic representations contained in the described figures are in no way representative of scale or proportions.

The methods described in detail in the foregoing and the illustrated devices are simply example embodiments that may be modified in the most diverse ways by the person skilled in the art without leaving the scope of the invention. Further, the use of the indefinite articles “a” or “an” does not rule out the possibility that the features in question may also be present more than once. Similarly, the terms “unit” and “element” do not rule out the possibility that the components in question may consist of a plurality of cooperating subcomponents, which, if necessary, may also be spatially distributed.

In the context of the present application, the expression “based on” may be understood, in particular, in the sense of the term “using.” In particular, a formulation according to which a first feature is produced (e.g., alternatively, ascertained, determined, etc.) based on a second feature does not rule out the possibility that the first feature may be produced (e.g., alternatively, ascertained, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for providing a comparison data set, the method comprising:
providing a first data set that is time-resolved and has a plurality of first data points, each first data point of the plurality of first data points having a time intensity curve, wherein the time intensity curves of the plurality of first data points map a first contrast agent flow in a region of interest of an examination subject in a first time period;
providing a second data set that is time-resolved and has a plurality of second data points, each second data point of the plurality of second data points having a time intensity curve, wherein the time intensity curves of the plurality of second data points map a second contrast agent flow in the region of interest of the examination subject in a second time period;
spatially and temporally registering the first data set and the second data set;
classifying areas of the region of interest mapped in the registered first data set and the registered second data set into first areas that comprise contrasted vessel segments, and second areas that comprise a tissue region;
identifying a deviation between the second areas of the registered first data set and the registered second data set based on a comparison of the time intensity curves of the registered first data set and the registered second data set that map the second areas; and
providing the comparison data set based on the registered first data set and the registered second data set,
wherein the comparison data set comprises at least one parameter characterizing the deviation.

2. The method of claim 1, wherein identifying the deviation comprises extracting a blood flow parameter from the respective time intensity curve.

3. The method of claim 2, wherein the extracting of the blood flow parameter from the respective time intensity curve is based on an area under the respective time intensity curve, a maximum intensity of the respective time intensity curve, a length of time from an increase in the intensity up to a maximum intensity of the respective time intensity curve, or any combination thereof.

4. The method of claim 2, wherein the identifying of the deviation comprises identifying an area by area blood flow change based on the extracted blood flow parameters, and
wherein the comparison data set is provided in a manner in which the comparison data set has at least one parameter characterizing the area by area blood flow change.

5. The method of claim 1, wherein the classifying of the areas comprises a vessel, tissue, or vessel and tissue segmentation in the registered first data set and the registered second data set.

6. The method of claim 5, wherein the identifying of the deviation comprises identifying an area by area blood flow change based on the extracted blood flow parameters,
wherein the comparison data set is provided in a manner in which the comparison data set has at least one parameter characterizing the area by area blood flow change,
wherein at least one proximal area of the region of interest mapped in the registered first data set and the registered second data set is identified based on the vessel segmentation and the area by area blood flow change,
wherein the time intensity curves of the proximal area exhibit a comparatively shortest time up to a maximum intensity in the vessel segmentation in an area exhibiting comparatively reduced blood flow, and
wherein the comparison data set is provided having an identification of the at least one proximal area.

7. The method of claim 1, wherein the spatial and temporal registration of the first data set and the second data set comprises a normalization of the time intensity curves of the first data set and the second data set.

8. The method of claim 1, wherein the identifying of the deviation comprises an identification, data point by data point, of a respective deviation between the plurality of first data points of the registered first data set and the plurality of second data points of the registered second data set.

9. The method of claim 1, wherein the providing of the comparison data set comprises providing a graphical representation based on the deviation using a visualization unit.

10. The method of claim 9, further comprising:
comparing the deviation, data point by data point, with a predefined threshold value; and
masking out the deviation, data point by data point, the graphical representation as a function of the comparison when the predefined threshold value is undershot or exceeded.

11. The method of claim 9, wherein the providing of the comparison data set comprises providing a graphical representation based on an at least partial overlaying of the deviation with a vessel representation of the examination subject.

12. The method of claim 1, wherein the providing of the comparison data set comprises providing an identification of vessels that are mapped in the first data set and not in the second data set, that are mapped in the first data set and not in the second data set, or a combination thereof.

13. A provisioning unit comprising:
a processor configured to provide a comparison data set, the processor being configured to provide the comparison data set comprising the processor being configured to:
provide a first data set that is time-resolved and has a plurality of first data points, each first data point of the plurality of first data points having a time intensity curve, wherein the time intensity curves of the plurality of first data points map a first contrast agent flow in a region of interest of an examination subject in a first time period;
provide a second data set that is time-resolved and has a plurality of second data points, each second data point of the plurality of second data points having a time intensity curve, wherein the time intensity curves of the plurality of second data points map a second contrast agent flow in the region of interest of the examination subject in a second time period;
spatially and temporally register the first data set and the second data set;
classify areas of the region of interest mapped in the registered first data set and the registered second data set into first areas that comprise contrasted vessel segments, and second areas that comprise a tissue region;
identify a deviation between the second areas of the registered first data set and the registered second data set based on a comparison of the time intensity curves of the registered first data set and the registered second data set that map the second areas; and
provide the comparison data set based on the registered first data set and the registered second data set,
wherein the comparison data set comprises at least one parameter characterizing the deviation.

14. A medical imaging device comprising:
a provisioning unit comprising:
a processor configured to provide a comparison data set, the processor being configured to provide the comparison data set comprising the processor being configured to:
provide a first data set that is time-resolved and has a plurality of first data points, each first data point of the plurality of first data points having a time intensity curve, wherein the time intensity curves of the plurality of first data points map a first contrast agent flow in a region of interest of an examination subject in a first time period;
provide a second data set that is time-resolved and has a plurality of second data points, each second data point of the plurality of second data points having a time intensity curve, wherein the time intensity curves of the plurality of second data points map a second contrast agent flow in the region of interest of the examination subject in a second time period;
spatially and temporally register the first data set and the second data set;
classify areas of the region of interest mapped in the registered first data set and the registered second data set into first areas that comprise contrasted vessel segments, and second areas that comprise a tissue region;
identify a deviation between the second areas of the registered first data set and the registered second data set based on a comparison of the time intensity curves of the registered first data set and the registered second data set that map the second areas; and
provide the comparison data set based on the registered first data set and the registered second data set,
wherein the comparison data set comprises at least one parameter characterizing the deviation, and
wherein the medical imaging device is configured to acquire, provide, or acquire and provide the first data set and the second data set.
